(19) United States
(12) Patent Application Publication
(10) Pub. No.: US 2012/0029393 A1
(43) Pub. Date: Feb. 2, 2012

(54) COMPACT ULTRASOUND TRANSDUCER ASSEMBLY AND METHODS OF MAKING AND USING THE SAME

(75) Inventor: Warren Lee, Niskayuna, NY (US)

(73) Assignee: GENERAL ELECTRIC COMPANY, SCHENECTADY, NY (US)

(21) Appl. No.: 12/847,041

(22) Filed: Jul. 30, 2010

(51) Int. Cl. A61N 7/00; H04R 31/00

(52) U.S. Cl. 601/2; 29/594

(57) ABSTRACT

A compact ultrasound transducer assembly is provided. The ultrasound transducer assembly comprises a compact housing, a therapy transducer disposed in the compact housing, and an interchangeable Fresnel lens having a flat surface and a structured surface. The Fresnel lens is disposed in the compact housing so that the flat surface of the Fresnel lens faces the therapy transducer. The structured surface of the Fresnel lens is disposed in a direction away from the therapy transducer, and the Fresnel lens is removably coupled to the therapy transducer, the compact housing, or both.

### BACKGROUND

The invention relates to an ultrasound transducer assembly, and more particularly to a compact ultrasound transducer assembly having adjustable focal depth, and methods of making and using the same.

Ultrasound refers to acoustic waves having a frequency above the upper limit of the human audible range (i.e., above 20 kHz). In ultrasonic applications (such as imaging and delivering therapy), acoustic waves are generated by a transducer and directed towards a region of interest. It is often desirable to focus the acoustic waves at different focal depths within a target (such as a patient). The different focal depths may be located within the same region of interest, or may be present in different regions of interest within the target.

Conventionally, while providing therapy, therapy transducers are mechanically focused using shaped piezoelectric transducers. Such transducers are typically spherical in shape and are bulky and expensive to design and manufacture due to the complexity involved in shaping and surface-finishing of non-flat piezoelectric materials. The spherical shape is necessary in order to focus the ultrasonic energy at a particular depth. For integrated imaging and therapeutic ultrasound transducers, it is desirable to locate the imaging transducer at the center of the therapy transducer. However, the depth of the sphere of the therapy transducer results in an increased distance between the apex and the contact face of the therapy transducer. Thus, for a spherical shaped transducer, the imaging transducer is located at a relatively long distance from the focal point of the therapy transducer, resulting in sub-optimal image quality. In addition, a spherical single element transducer has a fixed focal depth determined by the transducer geometry, i.e. it is not possible to change the focal depth. In order to vary the focal depth of a spherical transducer, it is possible to use an annular array of transducer elements, however, this greatly increases the cost and complexity of the transducer and system. Finally, the large dome-shaped transducer requires a large, bulky housing that is cumbersome to handle.

Therefore, it would be desirable to provide an ultrasound transducer assembly that provides a compact and cost-effective solution for adjusting a focal depth.

### BRIEF DESCRIPTION

In one embodiment, a compact ultrasound transducer assembly is provided. The ultrasound transducer assembly comprises a compact housing, a therapy transducer disposed in the compact housing, and an interchangeable Fresnel lens having a flat surface and a structured surface. The Fresnel lens is disposed in the compact housing so that the flat surface of the Fresnel lens faces the therapy transducer. The structured surface of the Fresnel lens is disposed in a direction away from the therapy transducer, and the Fresnel lens is removably coupled to the therapy transducer, the compact housing, or both.

In another embodiment, a compact ultrasound transducer assembly is provided. The ultrasound transducer assembly comprises a housing, a flat therapy transducer disposed in the housing, wherein the therapy transducer comprises a single element, an imaging transducer disposed in the housing, an interchangeable Fresnel lens that is removably coupled to the housing, or the therapy transducer, or both the housing and the therapy transducer, and a fastening structure that removably couples the Fresnel lens to the housing, or the therapy transducer, or both the housing and the therapy transducer.

In yet another embodiment, a method of assembling a compact ultrasound transducer assembly is provided. The method comprises providing a compact housing, disposing a therapy transducer in the compact housing, and removably coupling an interchangeable Fresnel lens to the therapy transducer, the compact housing, or both.

In another embodiment, a method for providing therapy to a region of interest is provided. The method comprises directing a first ultrasound beam to a region of interest at a first focal depth using a first interchangeable Fresnel lens, interchanging the first Fresnel lens with a second interchangeable Fresnel lens, wherein the interchangeable lenses comprise a fastening structure that enables the lenses to be readily detached and reattached without substantially interrupting the therapy, and directing another ultrasound beam at a second focal depth using the second interchangeable Fresnel lens.

### DRAWINGS

These and other features, aspects, and advantages of the invention will become better understood when the following detailed description is read with reference to the accompanying drawings in which like characters represent like parts throughout the drawings, wherein:

FIG. 1 is a cross-sectional view of an examples of an ultrasound transducer assembly comprising a layer of an adhesive material between the therapy transducer and an interchangeable Fresnel lens;

FIG. 2 is a cross-sectional view of an example of an ultrasound transducer assembly comprising recesses in the housing for receiving flanges formed in an interchangeable Fresnel lens;

FIG. 3 is a cross-sectional view of an example of an ultrasound transducer assembly comprising a housing having sockets arranged to receive protrusions in an interchangeable Fresnel lens;

FIG. 4 is a cross-sectional view of an example of an ultrasound transducer assembly comprising a housing having arms and an interchangeable Fresnel lens having protrusions;

FIG. 5 is a cross-sectional view of an example of an ultrasound transducer assembly comprising a housing having arms and an interchangeable Fresnel lens disposed on the arms of the housing;

FIG. 6 is a cross-sectional view of an example of an ultrasound transducer assembly comprising a housing having a fixed arm and a partially moveable arm;

FIG. 7 is a cross-sectional view of an example of an ultrasound transducer assembly comprising a housing and an interchangeable Fresnel lens having a coupler that is coupled to both the housing and the interchangeable Fresnel lens;

FIG. 8 is a cross-sectional view of an example of an ultrasound transducer assembly comprising a clip for coupling the housing to an interchangeable Fresnel lens;

FIG. 9 is a cross-sectional view of an example of an ultrasound transducer assembly comprising a housing and an interchangeable Fresnel lens having a spring loaded bearing for mounting the Fresnel lens to the housing;

FIG. 10 is a cross-sectional view of an example of an ultrasound transducer assembly comprising a housing and an interchangeable Fresnel lens with one or more thumbscrews to fasten the Fresnel lens to the housing;

FIG. 11 is a schematic diagram of an example of a method for assembling the ultrasound transducer assembly of the invention;

FIG. 12 is a schematic diagram of an example of a method for replacing one interchangeable lens with another; and

FIG. 13 is a schematic diagram of an example of an ultrasound system comprising ultrasound transducer assembly of the invention.

### DETAILED DESCRIPTION

The ultrasound transducer assemblies of the invention provide a simplified, inexpensive and compact device for delivering ultrasonic therapeutic energy at varying focal depths inside a target. In certain embodiments, the ultrasound transducer assembly comprises a compact ultrasound transducer assembly with a compact housing, a flat therapy transducer disposed in the compact housing and an interchangeable Fresnel lens disposed in the compact housing. The interchangeable Fresnel lens is removably coupled to the therapy transducer, the compact housing, or both. The compact ultrasound transducer assembly may also comprise an imaging transducer. In one embodiment, the therapy transducer may be a high intensity focused ultrasound (HIFU) transducer. In one example, the therapy transducer may operate at a frequency in a range from about 0.1 MHz to about 50 MHz.

The region of interest may be a one-dimensional, two-dimensional or a three dimensional region. The region of interest is typically located inside a patient. The region of interest may comprise undesirable tissue, such as diseased tissue or adipose tissue. In one example, providing the therapy may include destroying the undesirable tissue.

As used herein, the term “Fresnel lens” means an acoustically directing component through which acoustic energy may be passed to direct, or redirect the acoustic energy, for example, by focusing the acoustic energy to one or more foci. The Fresnel lens may have a flat surface and a structured surface opposite the flat surface. The structured surface may comprise a series of closely spaced grooves that control the refraction of acoustic energy. The acoustic energy from the ultrasound transducer is focused using the series of grooves. The grooves may act like concentric prisms that bend and focus the acoustic energy. The Fresnel lens may be a thin, and relatively flat structure.

In certain embodiments, the interchangeable Fresnel lens is disposed in the beam path of an ultrasonic beam propagating from the therapy transducer. The interchangeable Fresnel lens may be disposed in the ultrasound transducer assembly such that the flat surface of the interchangeable Fresnel lens is closer to the therapy transducer than the structured surface. An acoustic coupling medium may be disposed at least in a portion between the therapy transducer and the flat surface of the Fresnel lens to provide enhanced acoustic coupling between the therapy transducer and the Fresnel lens. Non-limiting examples of the acoustic coupling medium may include acoustic gel, or water. The acoustic coupling medium may be in the form of a thin layer of gel or water. While an acoustic coupling medium may be required between the therapy transducer and the Fresnel lens, there may not be a need for an acoustic coupling medium between the housing and the Fresnel lens. The acoustic coupling medium may be pre-disposed on the flat surface, the structured surface or both, of the Fresnel lens, before disposing the Fresnel lens in the ultrasound transducer assembly. For example, an acoustic coupling gel may be disposed on the flat surface of the Fresnel lens prior to coupling the Fresnel lens with the therapy transducer. Alternatively, the acoustic coupling medium may be disposed on the flat surface, the structured surface or both, of the Fresnel lens during or after disposing the Fresnel lens in the ultrasound transducer assembly.

The terms “interchangeable Fresnel lens” and “Fresnel lens” may be used interchangeably throughout the application. The interchangeable Fresnel lens may include an acoustic coupling medium disposed on the flat surface, the structured surface, or both. The interchangeable Fresnel lens may focus, shape, or direct the ultrasonic energy to a single focus or to a plurality of foci. The thickness of the interchangeable Fresnel lens may depend on the desired focal depth of the ultrasound beam and the number of grooves. In one embodiment, the thickness of the interchangeable Fresnel lens may be in a range from about 1 mm to about 20 mm. The interchangeable Fresnel lens may be made of a polymer material. In one example, the interchangeable Fresnel lens may be made of a flexible material. The interchangeable Fresnel lens may be formed using inexpensive methods such as but not limited to, casting or injection molding. The interchangeable Fresnel lens may have a low acoustic absorption.

Advantageously, the compact ultrasound transducer assembly enables the same therapy transducer to be used in applications that require focusing acoustic waves at different focal depths. The acoustic waves may be focused at different focal depths by simply replacing one interchangeable Fresnel lens with another. One interchangeable Fresnel lens may be replaced by another using simple techniques, such as but not limited to, simply unscrewing a lens and replacing it. Since a Fresnel lens used in a HIFU transducer may be exposed to potentially damaging effects of high intensity ultrasound, a method for easy replacement of an inexpensive Fresnel lens may provide cost savings for the user, who might otherwise require the transducer assembly to be serviced. Changing the Fresnel lens may also help to reduce the build up of heat on the patient and the Fresnel lens.

In addition, since the focal depth of the therapy transducer can be varied by using different Fresnel lenses, that is, since the transducer elements are not relied upon to vary the focal depth, the therapy transducer may be, but is not limited to, a single element transducer. The single element transducer simplifies the electrical system design and also reduces the manufacturing cost for the ultrasound transducer assembly. The single element transducer may use a single transmitting system (e.g. channel). Advantageously, single element transducer simplifies the system design and reduces the cost of the transducer assembly. Also, the compact shape of the therapy transducer and the Fresnel lens reduces the typical bulkiness of a spherical ultrasound transducer.

Thermal stresses that develop in the ultrasound transducer assembly may decouple the interchangeable Fresnel lens from the therapy transducer. In one embodiment, a thermally conductive layer may be provided between the therapy transducer and the interchangeable Fresnel lens to alleviate stresses arising in the transducer assembly due to the thermal expansion mismatch between the therapy transducer and the Fresnel lens. Alternatively, the acoustic coupling medium may itself serve the function of a thermally conductive layer.

In certain embodiments, an imaging transducer may be disposed in the compact housing, in addition to the therapy transducer. The flat shape of the therapy transducer enables the imaging transducer to be disposed closer to the focal depth of the acoustic waves of the therapy transducer than a spherical ultrasound transducer. The imaging signals are back-scattered from structures in the body, such as adipose tissue, muscular tissue, blood cells, veins or objects within the body (e.g., a catheter or needle), to produce echoes that return to the imaging transducer. The imaging transducer may receive the backscattered waves at different times, depending on the distance into the tissue from which they return and the angle at which they return. The interchangeable Fresnel lens may be coupled to one or more of the housing, the imaging transducer, or the therapy transducer.

In certain embodiments, the therapy or imaging transducers may comprise one or more transducer elements, one or more matching layers, and focusing components, such as an acoustic lens. The transducer elements may be arranged in a spaced relationship, such as, but not limited to, an array of transducer elements disposed on a layer, where each of the transducer elements may include a transducer front face and a transducer rear face. The transducer elements may comprise, but are not limited to, a piezoelectrically active material, such as lead zirconate titanate (PZT), lithium niobate, lead titanate, barium titanate, and/or lead metaniobate, or combinations thereof. The piezoelectrically active component of the transducer element may also, or alternatively, comprise one or more of a piezoelectric ceramic, a piezoelectric crystal, piezoelectric plastic, and/or piezoelectric composite materials. In addition to, or instead of, a piezoelectrically active material, transducer may comprise any other materials configured for generating radiation and/or acoustical energy such as capacitively coupled transducers or other acoustic sources. The transducer may also comprise one or more matching and/or backing layers coupled to the piezoelectrically active material. The therapy transducer may also include one or more matching layers disposed adjacent to the front face of the transducer elements, where each of the matching layers may include a matching layer front face and a matching layer rear face. The matching layers facilitate acoustic impedance matching of the differentials that may exist between the high impedance transducer elements and a low impedance patient.

Although, the embodiments illustrated hereinafter are described in the context of a medical imaging system, the compact ultrasound transducer assemblies may be adapted for other medical and non-medical applications, such as, for example, industrial borescopes that are used for monitoring and detection.

In certain embodiments, a fastening structure removably couples the interchangeable Fresnel lens to the therapy transducer, the compact housing, or both. The fastening structure may comprise a screw, key, latch, notch, socket, protrusion, removable adhesive, snap-fit, self-locking fit, clip, hook, pin, flange, coupling arm, deformable material, spring-loaded bearing, or combinations thereof.

The removable adhesive layer or a deformable material, such as a deformable substrate, may be disposed between the therapy transducer and the Fresnel lens. In one example, the fastening structure may be configured to act as an acoustic coupling medium. For example, the adhesive layer or the deformable substrate may be made of a material that can enhance the acoustic coupling between the therapy transducer and the Fresnel lens. The adhesive or deformable substrate may have low acoustic attenuation. The adhesive material or the deformable material may be present in the form of a continuous layer or as a combination of a plurality of discrete spots. In another example, a portion or the entire housing may be made of a deformable material. In this example, the housing may elastically or plastically deform upon receiving the Fresnel lens. The elastic or plastic deformation area in the housing material may only be a few microns in size. This deformation may provide a sealing between the housing and the Fresnel lens to hold the Fresnel lens in place.

The deformable substrate may be a flexible substrate. The material of the deformable substrate may be configured to undergo elastic or plastic deformation when receiving the Fresnel lens. In certain embodiments, the material for the deformable substrate may be such that a recess can be formed in the deformable substrate. The recess may be dimensioned to receive the interchangeable Fresnel lens. In some embodiments, the deformable substrate may include polymers. The material of the deformable substrate may be chosen based on the ease of forming the desired recess shape in the substrate material. The material of the deformable substrate transmits a majority of the ultrasound energy from the transducer to the Fresnel lens. In one embodiment, the deformable substrate may include soft polymers. Soft polymers refer to elastomer type materials such as, but not limited to, polydimethylsiloxane, a copolymer of hexafluoropropylene (HFP) and vinylidene fluoride (VDF or VF₂), a terpolymer of tetrafluoroethylene (TFE), vinylidene fluoride (VDF), and hexafluoropropylene (HFP), perfluoromethylvinylether (PMVE), nitrile rubber, polyethylene, polymethylpentene, and thermoplastic elastomers such as ELASTRON® and THERMOLAST®.

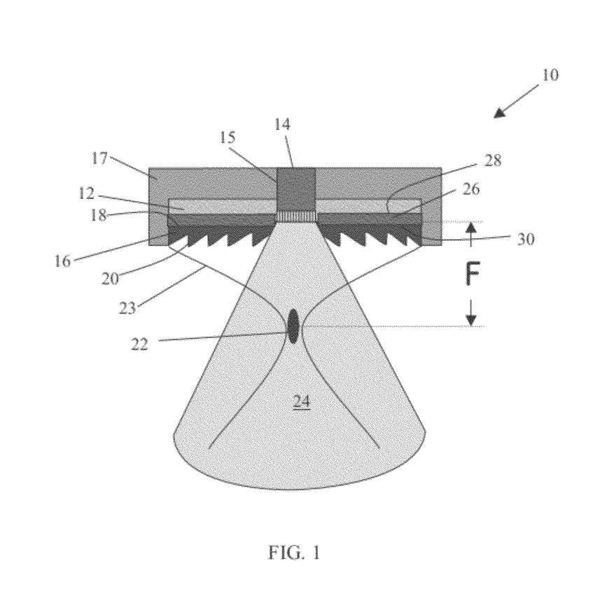

An embodiment of the compact ultrasound transducer assembly of the invention is generally shown and referred to in FIG. 1 as assembly 10. The assembly 10 comprises a therapy transducer 12, an imaging transducer 14, a shaft 15 for the imaging transducer 14, and an interchangeable Fresnel lens 16 disposed in a compact housing 17. The therapy transducer 12 is substantially flat in shape. The therapy transducer 12 may be a single element transducer. The therapy transducer 12 may generate high intensity focused ultrasound (HIFU). That is, the therapy transducer 12 may be a HIFU transducer. The interchangeable Fresnel lens 16 comprises a flat surface 18 that is disposed closer to the therapy transducer 12. The structured surface 20 of the Fresnel lens 16 may be disposed away from the therapy transducer. F represents the focal depth of the HIFU focal spot 22 of the therapy transducer 12 in the presence of the Fresnel lens 16. The focal depth F may be changed by replacing the Fresnel lens 16 with another Fresnel lens. The imaged field or ultrasound beam is represented by 24. Due to the flat shape of the therapy transducer 12, the imaging transducer 14 is disposed closer to the HIFU focal spot 22 than conventional spherical shaped therapy transducers, thereby improving the quality of the acquired image.

In this embodiment, a layer 26 of the adhesive material is used between the therapy transducer 12 and the Fresnel lens 16. The adhesive layer 26 may be a patterned or a continuous layer. The adhesive layer 26 may be a dual sided adhesive layer that has adhesive on both the sides. The side 28 of the adhesive layer may have a permanent adhesive, whereas the side 30 may have an adhesive that allows the Fresnel lens to be decoupled from the adhesive layer without having to remove the adhesive layer 26. Alternatively, the adhesive layer 26 may be a removable adhesive layer 26 that is part of the Fresnel lens 16. The side 30 of the adhesive layer 26 may be permanently coupled to the Fresnel lens and the side 28 may have a removable adhesive. Suitable temporary adhesives for removable side of the adhesive layer 26 may include epoxy based adhesives, thermoset adhesives, acrylate based adhesives, silicone-based adhesives, elastomer based adhesives, or any combinations thereof. Suitable permanent adhesives may be used on the other side.

In one embodiment, the imaging transducer 14 may be built into a central region of the therapy transducer 12. In this embodiment, the imaging transducer 14 may be permanently fixed to the central region of the therapy transducer. The imaging transducer may image through the central region of the Fresnel lens and may be designed so as to avoid distortions in the steering and focusing of the imaging beams. In another embodiment, imaging transducer 14 may be removably inserted into an opening in the therapy transducer 12. In this embodiment, the imaging transducer 14 may extend into the shaft 15. If the imaging transducer 14 extends past the surface of the therapy transducer 12, the sides of the imaging transducer 14 may obstruct part of the therapy beam 23. Conversely, if the imaging transducer 14 does not extend beyond the therapy transducer 12 (and lies flush with the therapy transducer 12), the opening in the Fresnel lens 16, to accommodate the shaft 15, is designed so that the opening does not obstruct part of the imaging field of view 24 of the imaging transducer 14. An acoustic coupling medium may be disposed between the imaging transducer 24 and the cavity for the shaft 15.

Although not illustrated, the hole for the shaft 15 may extend to the structured surface 20 of the Fresnel lens 16. In this case, the Fresnel lens 16 may have a minimal impact on the focusing and steering of the ultrasound beam from the imaging transducer in that region. An insert may be disposed in the central region of the Fresnel lens 16, where the insert may be made of an acoustically neutral material, and the imaging transducer may image through this acoustically neutral material.

In one embodiment, the therapy transducer 12 may rotate or oscillate about an axis. In this embodiment, for the ease of assembly, the shaft 15 for the imaging transducer 14 may coincide with the central axis of the therapy transducer 12.

In one embodiment, the compact housing comprises a receiving notch or a detent. The Fresnel lens comprises a ledge, flange, pin, or projection that engages the housing receiving detent or socket and holds the Fresnel lens. The Fresnel lens may be partially movable between a first position where the Fresnel lens is fastened to the housing and a second position where the Fresnel lens is removable from the housing.

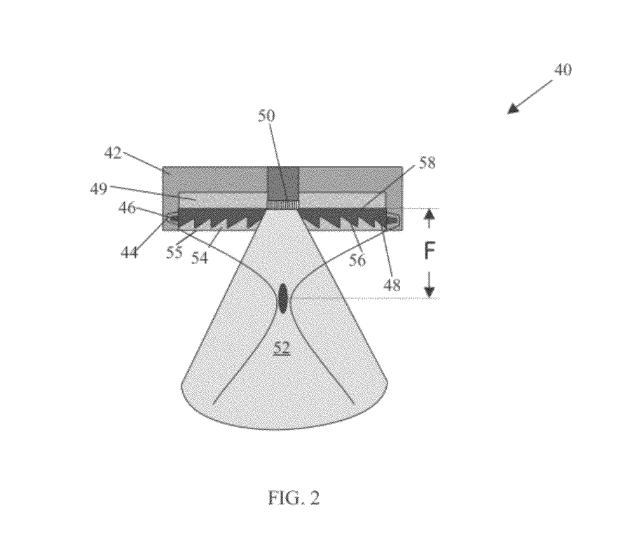

FIG. 2 illustrates another embodiment of the compact ultrasound transducer assembly 40 where the housing 42 comprises recesses 44 for receiving flanges 46 formed in the Fresnel lens 48. The therapy transducer 49 comprises a focal depth F and the imaging transducer 50 comprises field of view 52. Although not illustrated, an acoustic coupling medium may be disposed between the flat surface 58 of the Fresnel lens 48 and the therapy transducer 49. The Fresnel lens 48 further comprises a layer 54 of an acoustic coupling medium present on the structured surface 56 of the Fresnel lens 48. The layer 54 of the coupling medium may be enclosed by a thin membrane 55. In one embodiment, an acoustic coupling medium may be applied to the thin membrane 55 or the layer 54 (if the thin membrane 55 is not present) to avoid air at the interface between the ultrasound transducer assembly 40 and the patient.

If the grooves on the structured surface 56 are deep and have sharp edges, the layer 54 may have sufficient thickness to avoid any discomfort to a patient that may be otherwise caused due to the sharp grooves. That is, the layer 54 may be used to smoothen the structured surface 56 so that there is no discomfort caused to the patient due to the sharp grooves of the structured surface 56.

Alternatively, if the structured surface 56 of the Fresnel lens comprises grooves that are shallow, and the edges of the grooves are not very sharp, the layer 54 of the coupling medium may not be required on the structured surface 56. However, to enhance acoustic coupling between the patient and the ultrasound transducer assembly and to avoid air at the interface, an acoustic coupling medium may be applied to the structured surface 56 prior to providing treatment to the patient. The acoustic coupling medium so applied may form a thin conformal coating on the grooves of the structured surface 56, and may have a relatively low thickness as compared to the layer 54 of the acoustic coupling medium. In one example, the acoustic coupling medium may be applied immediately prior to providing treatment to the patient.

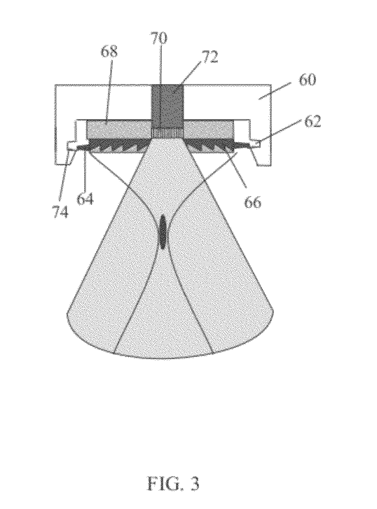

FIG. 3 illustrates a housing 60 having sockets 62 arranged to receive protrusions 64, such as but not limited to circular, square or rectangular protrusions, of a Fresnel lens 66. A therapy transducer 68, an imaging transducer 70, and a shaft 72 are also disposed in the housing 60. The sockets 62 comprise shoulder 74, onto which the Fresnel lens 66 is snapped by gently deflecting the Fresnel lens 66 over the shoulder, so that the protrusions 64 are disposed into the sockets 62 and rest on the shoulder 74. To decouple the Fresnel lens 66 from the sockets 62, one of the protrusions 64 may be further pushed inside the corresponding socket 62, and the other protrusion may be guided out of the socket. One or both of the sockets 62 may be long enough to allow the protrusions 64 of the Fresnel lens 66 to slide within the sockets 62.

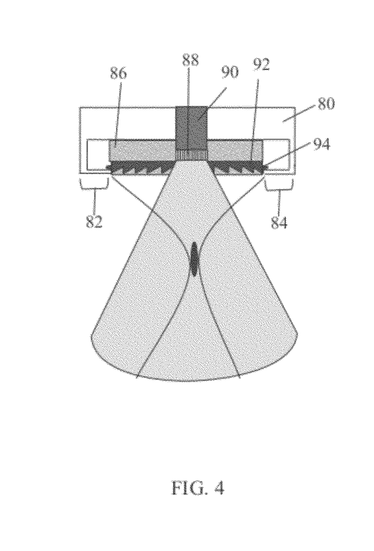

FIG. 4 illustrates an embodiment where a housing 80 comprises a bracket in which the two arms 82 and 84 of the bracket may have the same or different lengths. A therapy transducer 86, an imaging transducer 88 and a shaft 90 for the imaging transducer 88 are disposed within the housing 80.

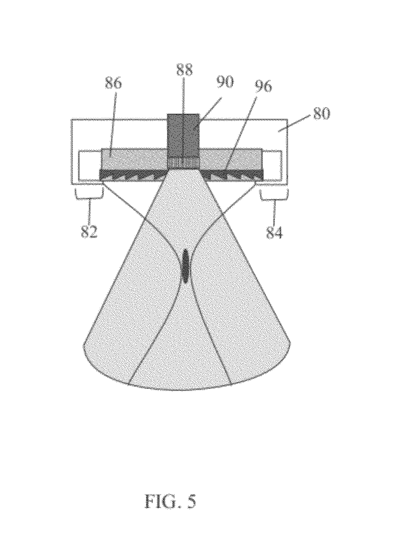

A Fresnel lens 92 may have small protrusions 94 to enable the Fresnel lens 92 to be disposed within the housing 80. Alternatively, as illustrated in FIG. 5 a Fresnel lens 96 may be disposed on the arms 82 and 84 of the housing. In this embodiment, the Fresnel lens 96 may not include protrusions 94.

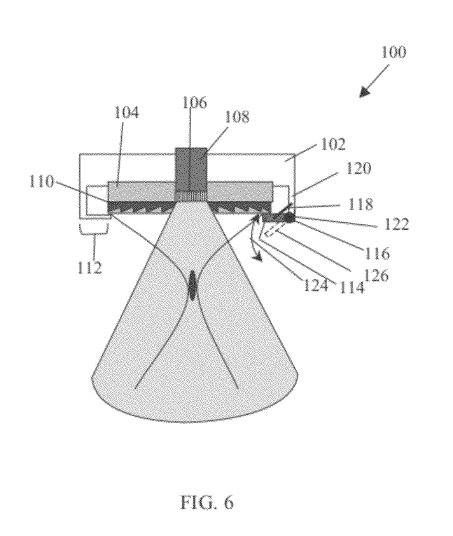

FIG. 6 illustrates a compact ultrasound transducer assembly 100 having a housing 102 for disposing a therapy transducer 104, an imaging transducer 106, a shaft 108 for the imaging transducer 106, and a Fresnel lens 110. The housing 102 comprises a fixed arm 112 and a partially moveable arm 114. The arm 114 is hinged at one end 116. The arm 114 further comprises a latch, represented by a line 118. The latch extends between the arm 114 and leg 120 of the housing 102. The latch 118 is configured to be decoupled from the leg 120 to allow the arm 114 to rotate about the hinge 122, as represented by a curved arrow 124 and a dashed rectangle 126. The arm 114 may be released to the position 126 when the Fresnel lens 110 needs to be replaced. After replacing the Fresnel lens 110, the arm 114 may be moved to the original position (solid rectangle 114), and the arm 114 may be secured in its place using the latch 118. The Fresnel lens 110 may or may not have small projections. Also, in one embodiment, both the arms 112 and 114 may be moveable.

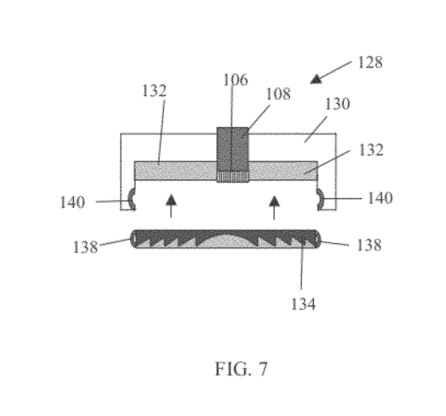

FIG. 7 illustrates a compact ultrasound transducer assembly 128 having a housing 130 for a therapy transducer 132, an imaging transducer 106, and a shaft 108 for the imaging transducer 106. The housing 130 and a Fresnel lens 134 comprise a coupler that is coupled to both the housing 130 and the Fresnel lens 134. The coupler comprises a snap having male couplers 138 and female couplers 140. The male couplers 138 are formed on the Fresnel lens 134 and the female couplers 140 are formed on the housing 130. Alternatively, male couplers 138 may be formed on the housing 130 and the female couplers 140 may be formed on the Fresnel lens 134. Also, other means for removably coupling the housing 130 and the Fresnel lens 134 may be used in place of male and female couplers 138 and 140. For example, couplers 138 and 140 may comprise threaded screws and corresponding threads in the housing, a dowel and pin assembly, a lock and key assembly, a post and clasp assembly, or a hook and loop fastener.

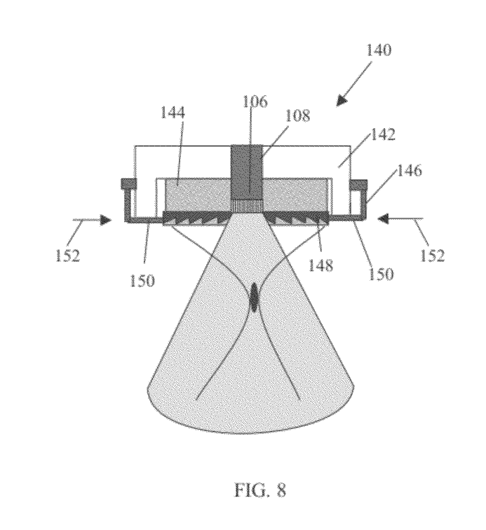

FIG. 8 illustrates a compact ultrasound transducer assembly 140 having a housing 142 for a therapy transducer 144, an imaging transducer 106, and a shaft 108 for the imaging transducer 106. A clip 146 is used to couple the housing 142 to the Fresnel lens 148 by snapping over the end of the housing 142 and the perimeter of Fresnel lens 148. Alternatively, the clip 146 may be snapped through a groove in the housing 142. The clip 146 may have a “U” or semicircular shape, and the clip 146 has flanges 150 that engage the Fresnel lens 148. The Fresnel lens 148 may have grooves to receive the flanges 150. The clip 146 may also engage the Fresnel lens 148 by pushing it toward the Fresnel lens 148 and in a direction transverse to a centerline of the Fresnel lens 148 as represented by arrows 152. The Fresnel lens 148 may be removed by pulling the clip in a direction opposite to that shown by the arrows 152. The clip 146 may be made of a material that retains its resilience after being repeatedly subjected to expansion (while removing the Fresnel lens 148). Such materials include, but are not limited to, spring steel and stainless steel.

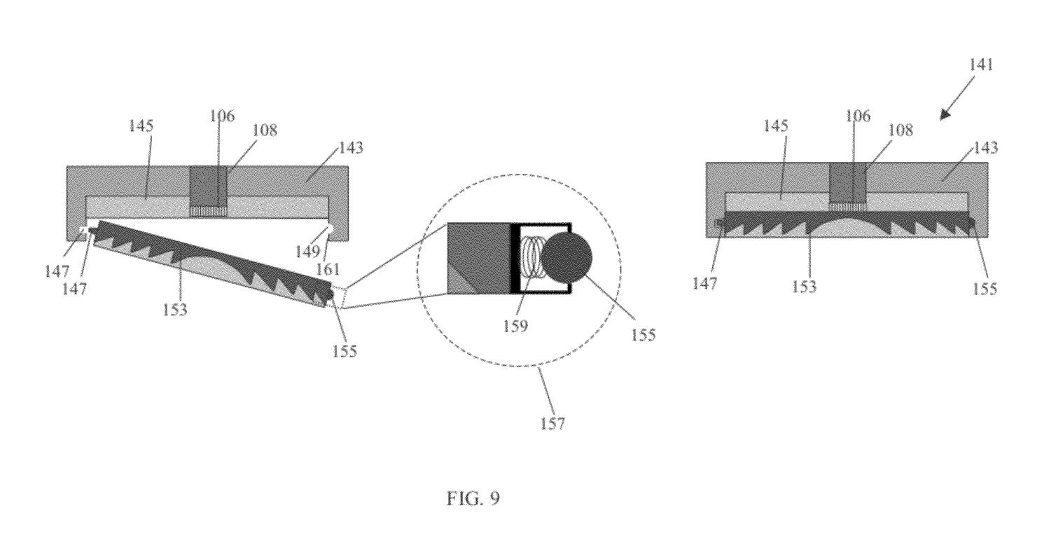

FIG. 9 illustrates a compact ultrasound transducer assembly 141 having a housing 143 for a therapy transducer 145, an imaging transducer 106, and a shaft 108 for the imaging transducer 106. The housing 143 comprises recesses 147 and 149. Although, in the illustrated embodiment, the recess 147 is shown as a cylindrical recess, and the recess 149 is shown as a spherical recess 149, however, these recesses 147 and 149 may have any other suitable shapes. The cylindrical recess 147 is configured to receive protrusion 151 of an interchangeable Fresnel lens 153. The spherical recess 149 is configured to receive a spring-loaded spherical bearing 155 of the Fresnel lens 153. As illustrated in the enlarged view in the dashed circle 157, the spring-loaded spherical bearing 155 comprises a spring 159 that is coupled to the Fresnel lens 153. During installation of the Fresnel lens 153 in the transducer assembly 141, the bearing 155 is compressed into the housing 143 upon encountering the shoulder 161 of the housing 143. Upon installation of Fresnel lens 153 in the housing 143 the bearing 155 is disposed into the spherical recess 149 of the housing 143. The spring loaded bearing 155 may be at one or more positions around the perimeter of the Fresnel lens 153. Although not illustrated, a lever arm may be provided in the housing 143 to ease the removal of the Fresnel lens 153.

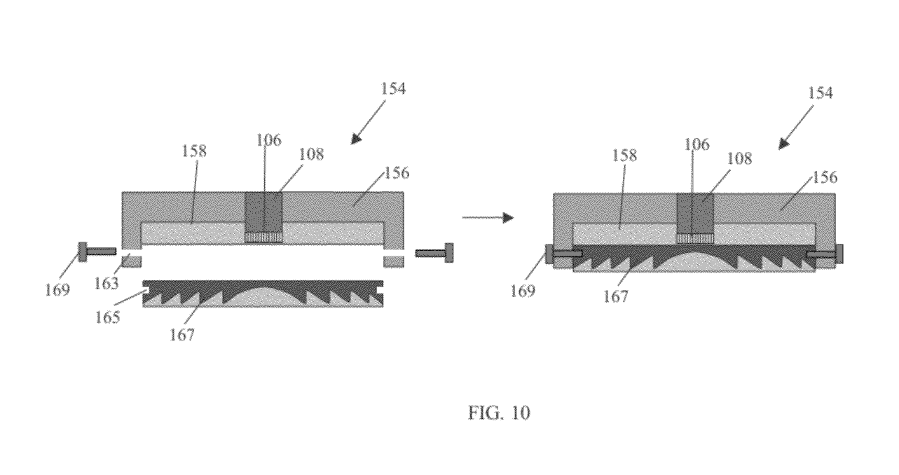

FIG. 10 illustrates a compact ultrasound transducer assembly 154 having a housing 156 for a therapy transducer 158, an imaging transducer 106, and a shaft 108 for the imaging transducer 106. The housing 156 comprises two or more recesses 163 in the housing 156, and recesses 165 in an interchangeable Fresnel lens 167. One or both of the recesses 163 and 165 may be threaded. Screws, such as thumbscrews 169 may be inserted in the recesses 163 and 165 to couple Fresnel lens 167 to the housing 156. Although not illustrated, the thumbscrews 169 may be replaced with capture pins or clips that are coupled to the housing 156.

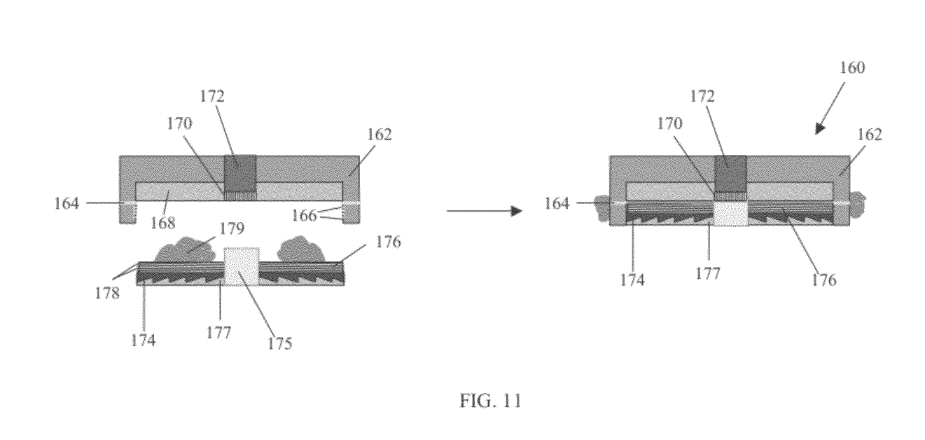

FIG. 11 is a schematic drawing of a method for assembling a compact ultrasound transducer assembly 160. In this embodiment, a housing 162 comprises passageways 164 and threads/notches 166. The passageways 164 may be used for releasing excess air, acoustic coupling medium, or excess adhesive, from the transducer assembly. A therapy transducer 168 and an imaging transducer 170 are disposed in the housing 162. The imaging transducer 170 is coupled to a shaft 172. The therapy transducer 168 may also be coupled to the shaft 172. Shaft 172 may also be coupled to a drive motor to rotate the imaging and/or therapy transducers 168 and 170.

Assembly 160 comprises Fresnel lens 174 having a channel 175 through which the shaft 172 passes. The Fresnel lens 174 is coupled to a structure 176 having pins/screws 178 for coupling the Fresnel lens to the housing 162. Other fastening structures may be used for coupling the Fresnel lens 174 to the housing 162. When the Fresnel lens 174 is coupled to the housing 162, the pins/screws 178 are received by the threads/notches 166. The structure 176 may be made of a glass, ceramic, metal, cermets, or polymers, such as but not limited to, thermoplastic elastomers, hard polymers, such as but not limited to, polyether ether ketone (PEEK), polypropylene, poly(methyl methacrylate) (PMMA), polyethelene, olefin copolymers, modified ethylene-tetrafluoroethylene) fluoropolymer (ETFE), polyetherimide, polyvinyl chloride (PVC), and the like.

The shaft 172 extends through the Fresnel lens 174 and may be used for advancing or withdrawing the imaging transducer 170 through the therapy transducer 168 and the Fresnel lens 174.

The Fresnel lens 174 comprises acoustic coupling medium 177 disposed on the structured surface of the Fresnel lens 174. An acoustic coupling medium 179 (such as gel) may be disposed on the structure 176 and the Fresnel lens 174, along with the structure 176, may be coupled to the housing 162. The excess acoustic coupling medium 178 may be released through the passageways 164 and removed simply by wiping off the excess. The acoustic coupling medium 179 provides acoustic coupling between the Fresnel lens 174 and the therapy transducer 168.

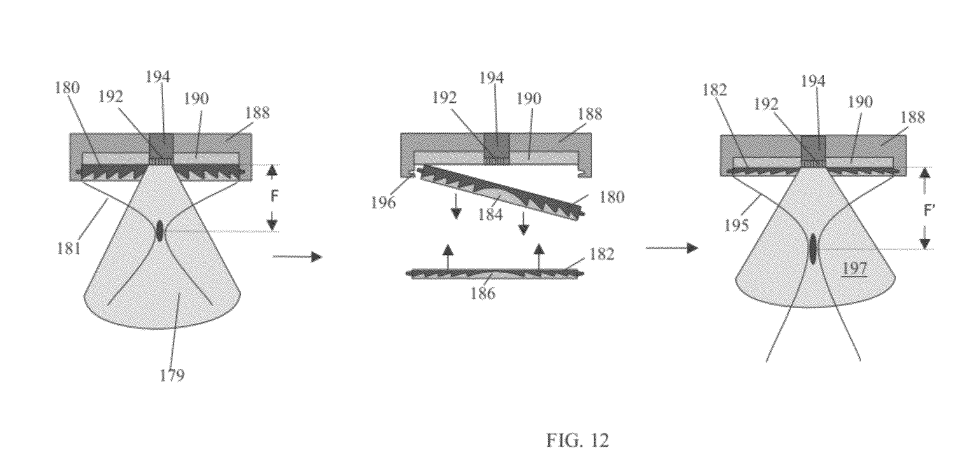

FIG. 12 is a schematic drawing of a method for quickly replacing one Fresnel lens with another without significantly interrupting a therapy procedure. The therapy beam 181 is directed to a region of interest at a focal length F using a first interchangeable Fresnel lens 180. The imaging field 179 includes the HIFU focus of the therapy beam 181. Subsequently, the first interchangeable Fresnel lens 180 is replaced with a second interchangeable Fresnel lens 182 having a second focal length F′, which is different from the first focal length F. A second therapy beam 195 is directed to same or different region of interest at the second focal depth F′ using the second interchangeable Fresnel lens 182. The imaging field 197 includes the HIFU focus of the therapy beam 195. The first and second Fresnel lenses 180 and 182 have acoustic coupling media 184 and 186, respectively, predisposed on the structured surfaces of the Fresnel lenses 180 and 182. A therapy transducer 190, an imaging transducer 192, and a shaft 194 are disposed in a housing 188. In this embodiment, the housing 188 comprises slots 196 for coupling the Fresnel lenses 180 and 182 to the housing.

The Fresnel lenses 180 and 182 may be installed and removed by hand or with simple tools. A suitable removal tool would be capable of securely holding the housing 188 while allowing the user to decouple the Fresnel lens 180 from the housing 188. Such an assembly tool would be capable of securely holding the housing 188 while allowing the user to push the Fresnel lens 182 in the housing 188. In one embodiment, the housing 188 may be slightly expandable to detach the Fresnel lens from the housing 188.

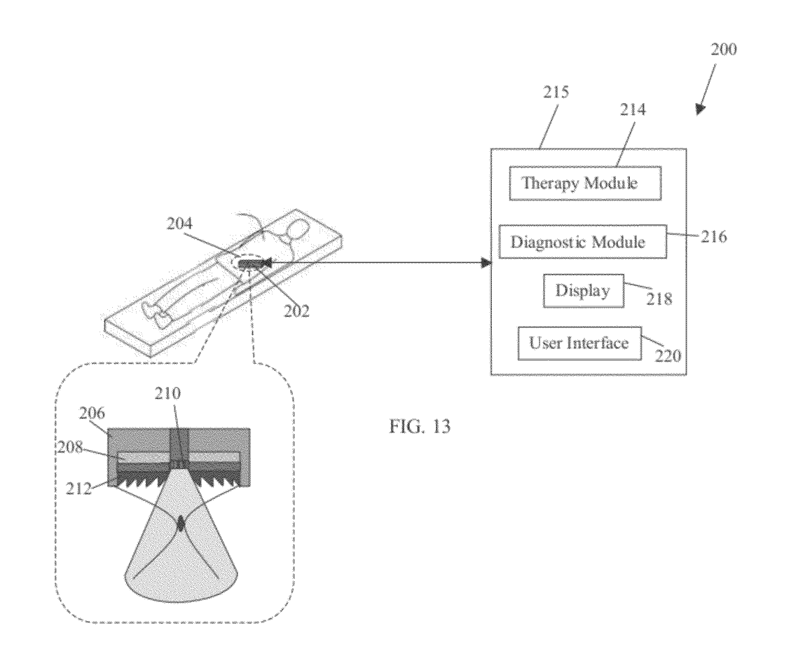

FIG. 13 is a schematic drawing of an example of a compact ultrasound system 200 having an ultrasound transducer assembly 202 that can provide therapy to the region of interest 204, and optionally, display images of the region of interest 204. The ultrasound transducer assembly 202 comprises a compact housing 206 for a therapy transducer 208, an imaging transducer 210, and an interchangeable Fresnel lens 212. In one example, the therapy transducer 208 may be used for ablating tissues, such as diseased tissues, adipose tissues, or non-adipose tissues. The imaging transducer 210 may image the region of interest 204 before applying the therapy, after applying the therapy, or while applying the therapy.

In addition to, or instead of, a piezoelectrically active material, transducers 208 and 210 may comprise other materials suitable for generating radiation and/or acoustic energy such as capacitively coupled transducers or other acoustic sources. Transducers 208 and 210 may also comprise one or more matching and/or backing layers coupled to the piezoelectrically active material. Transducers 208 and 210 may also comprise single or multiple damping elements.

A subsystem 215 may comprise a therapy module 214 for controlling the delivery of therapy to the treatment locations based on one or more therapy parameters or information from the diagnostic module. The therapy module 214 is connected to a user interface 220, such as a mouse or keyboard, and controls operation of the transducer. The therapy module 214 is configured to receive inputs from a user using the user interface 220. The therapy module 214 may receive imaging and/or therapy commands from the user through a user interface 220 for applying therapy to the region of interest 204.

The therapy module 214 may be implemented utilizing any combination of dedicated hardware boards, digital signal processors, and processors. While the therapy module 214 is configured to deliver a therapy to the treatment locations based on one or more therapy parameters or information from the diagnostic module, the diagnostic module 216 is configured to control the imaging transducer 210 to obtain diagnostic ultrasound signals from the region of interest 204.

A processing unit processes the acquired ultrasound information (e.g., RF signal data or IQ data pairs) and prepares frames of ultrasound information for display device 218. The display device 218 may comprise one or more monitors that may present patient information, such as diagnostic and therapeutic ultrasound images, to the user for review, diagnosis, analysis, and/or treatment. The display device 218 may automatically display, for example, a (two dimensional) 2D, (three dimensional) 3D, or (four dimensional) 4D ultrasound data set stored in the memory or currently being acquired. This stored data set may also be displayed with a graphical representation (e.g., an outline of a treatment space or a marker within the treatment space).

The flat therapy transducer with low profile Fresnel lens allows the imaging transducer to be located closer to the HIFU focus, improving image quality. In addition, the flat therapy transducer (e.g., HIFU) is much simpler and less expensive to manufacture than a spherical transducer. Also, the transducer assembly employing the interchangeable Fresnel lens is relatively simple and economical to manufacture, and may be manufactured using a batched and/or automated process.

Ease in replacing a Fresnel lens with another enables the user (such as the technician or physician operating the device) to replace the Fresnel lens in a time efficient fashion, and allows the physician to use a single transducer with different Fresnel lenses to accommodate a wide variety of patients and procedures. Since a primary cause of failure in a HIFU device is due to the HIFU energy damaging the materials in the acoustic path, inexpensively replacing one Fresnel lens with another avoids costly service and repairs to the transducer assembly.

While only certain features of the invention have been illustrated and described herein, many modifications and changes will occur to those skilled in the art. It is, therefore, to be understood that the appended claims are intended to cover all such modifications and changes as fall within the scope of the invention.

1. A compact ultrasound transducer assembly, comprising:
a compact housing;
a therapy transducer disposed in the compact housing; and
an interchangeable Fresnel lens having a flat surface and a structured surface, wherein the Fresnel lens is disposed in the compact housing so that the flat surface of the Fresnel lens faces the therapy transducer, wherein the structured surface of the Fresnel lens is disposed in a direction away from the therapy transducer, and wherein the Fresnel lens is removably coupled to the therapy transducer, the compact housing, or both.

2. The compact ultrasound transducer assembly of claim 1, further comprising a fastener that removably couples the Fresnel lens to the therapy transducer, the compact housing, or both.

3. The compact ultrasound transducer assembly of claim 1, wherein the therapy transducer comprises a single element transducer.

4. The compact ultrasound transducer assembly of claim 1, wherein the interchangeable Fresnel lens comprises an acoustic coupling medium pre-disposed on the flat surface, the structured surface, or both.

5. The compact ultrasound transducer assembly of claim 1, wherein the therapy transducer generates high intensity focused ultrasound.

6. The compact ultrasound transducer assembly of claim 1, wherein the Fresnel lens comprises a flexible material.

7. The compact ultrasound transducer assembly of claim 1, wherein the therapy transducer is a flat transducer.

8. The compact ultrasound transducer assembly of claim 1, wherein the compact housing comprises one or more through passageways for venting air or releasing excess acoustic coupling medium, or both.

9. The compact ultrasound transducer assembly of claim 1, wherein the interchangeable Fresnel lens has a thickness between 1 mm and 20 mm.

10. The compact ultrasound transducer assembly of claim 1, further comprising an imaging transducer.

11. The compact ultrasound transducer assembly of claim 10, wherein the imaging transducer is removably disposed through a shaft in the housing.

12. The compact ultrasound transducer assembly of claim 11, wherein the interchangeable Fresnel lens comprises a channel for the shaft into which the imaging transducer is disposed.

13. The compact ultrasound transducer assembly of claim 11, wherein the Fresnel lens is coupled to the shaft.

14. The compact ultrasound transducer assembly of claim 11, wherein the therapy transducer is coupled to the shaft.

15. A compact ultrasound transducer assembly, comprising:
a housing,
a flat therapy transducer disposed in the housing, wherein the therapy transducer comprises a single element;
an imaging transducer disposed in the housing;
an interchangeable Fresnel lens that is removably coupled to the housing, or the therapy transducer, or both the housing and the therapy transducer; and
a fastening structure that removably couples the Fresnel lens to the housing, or the therapy transducer, or both the housing and the therapy transducer.

16. A method of making a compact ultrasound transducer assembly, comprising:
providing a compact housing;
disposing a therapy transducer in the compact housing; and
removably coupling an interchangeable Fresnel lens to the therapy transducer, the compact housing, or both.

17. The method of claim 16, further comprising disposing an acoustic coupling medium between the therapy transducer and the interchangeable Fresnel lens.

18. The method of claim 16, wherein the compact housing comprises a fastener.

19. A method for providing therapy to a region of interest, comprising:
directing a first ultrasound beam to a region of interest at a first focal depth using a first interchangeable Fresnel lens;
interchanging the first interchangeable Fresnel lens with a second interchangeable Fresnel lens, wherein the interchangeable lenses comprise a fastening structure that enables the lenses to be readily detached and reattached without substantially interrupting the therapy; and
directing another ultrasound beam at a second focal depth using the second interchangeable Fresnel lens.
